(19) United States
(12) Patent Application Publication
(10) Pub. No.: US 2014/0004562 A1
(43) Pub. Date: Jan. 2, 2014

(54) REVERSIBLY WATER-SOLUBLE NANOCRYSTALS

(71) Applicants: Xinhua Zhong, Shanghai (CN); Lu Liu, Shanghai (CN)

(72) Inventors: Xinhua Zhong, Shanghai (CN); Lu Liu, Shanghai (CN)

(73) Assignee: EAST CHINA UNIVERSITY OF SCIENCE AND TECHNOLOGY, Shanghai (CN)

(21) Appl. No.: 13/878,358

(22) PCT Filed: Jan. 20, 2012
(86) PCT No.: PCT/CN2012/070659

§ 371 Date: Apr. 8, 2013

(51) Int. Cl. G01N 33/50

(52) U.S. Cl. CPC G01N 33/5005
USPC 435/40.5; 435/366; 435/370

(57) ABSTRACT

A general, facile, and reversible nanocrystal (NCs) phase transfer protocol via ligand exchange using nucleotides and/or nucleosides is provided to generate reversibly water-soluble nanocrystals. This phase transfer strategy can be employed on a wide variety of chemically synthesized nanostructured materials including semiconductors, metal oxides and noble metals with different sizes and shapes. The nucleotide/nucleoside-capped nanocrystals can disperse homogeneously in aqueous or alcohol media retaining, for example, high photoluminescence quantum yields. The disclosed water-soluble nanocrystals have excellent colloidal and photoluminescent stability independent on the pH and ionic strength, minimal hydrodynamic size, and are stable in cells and suitable for in vitro cell labeling, cell tracking, and other bioimaging applications.

### BACKGROUND

During the past decade, colloidal nanocrystals (NCs) have found numerous applications in optoelectronics, photovoltaic cells, catalysis, and biotechnology, due to their distinguished size- and shape-dependent properties. The innately high surface area-to-volume ratio of NCs results in the surface containing a large fraction of unsaturated atoms. For stabilization and functionalization purposes, organic surfactants are typically adsorbed onto the surface of NCs as a “cap” to passivate the dangling bonds. The capping organic ligands/surfactants stabilize the NCs dispersion and determine the physicochemical properties of the nanoparticles, such as the hydrodynamic size, toxicity, charge and intermolecular/interparticle interactions. The NC-organic surfactant interface plays an important role in NC structure and optoelectronics, therefore the ability to engineer surface properties of NCs is important for various applications.

Most chemically synthetic routes to high-quality NCs predominantly employ carboxylic acids, amines, or phosphine oxides with long hydrocarbon chains as capping ligands, which sterically stabilize NCs in hydrophobic solvents. However, the presence of such bulky capping molecules creates an insulating bather around each NC and blocks the access of molecular species to the NC surface, which is detrimental for electronic and catalytic applications. In addition, biological applications, such as cell- or organelle-staining, generally require NCs to be water-soluble and biocompatible, for which traditional hydrophobic, ligand-capped NCs are not suitable.

To address such specific applications, transfer of NCs from a hydrophobic environment to a hydrophilic one, or vice versa, is required in order to maximize the respective advantages of these environments. This makes phase transfer an important aspect in the functionalization and application of nanostructured materials. Typical phase transfer of NCs is achieved by replacing the original ligands with specifically designed molecules through a ligand-exchange process, or by cross-linking of NCs with a shell of silanols, or amphiphilic copolymers. Although surface modification based on ligand-exchange reactions has been actively explored in various NC systems, a generalized and efficient strategy is far from developed. Thiol-functionalized carboxylic acid ligands such as mercaptopropionic acid (MPA), dihydrolipoic acid (DHLA) are most often been employed for ligand exchange reaction. The thiol-functionalized ligands capped NCs are not stable enough due to the facile oxidization feature of the thiol group. In addition, due to the strong binding ability of the thiol group, this ligand-exchange process is typically irreversible, making it difficult to further functionalize NCs. To date, all reported reversible phase transfer approaches suffer from one or more of the following problems: i) the phase transfer process will deteriorate physicochemical properties of NCs, such as colloidal stability and optical properties; ii) the process only works for certain NC systems; iii) NCs may be dispersed in either polar or nonpolar solvent, but not truly and repeatedly reversible between aqueous and organic media; iv) the phase transfer reagents require tedious synthesis and are expensive.

Accordingly, there remains a need to develop a generalized, facilely reversible phase transfer methodology and materials for functionalized NCs between aqueous and organic phases.

### SUMMARY

A general ligand-exchange methodology is provided using nucleotides and/or nucleosides to replace the initial hydrophobic ligands (with long hydrocarbon tails) on the surface of NCs, thus rendering the initial oil-soluble NCs reversibly water-soluble. A reversibly water-soluble nanocrystal comprising a core nanocrystal capped by a plurality of nucleotides, a plurality of nucleosides, or a combination thereof is provided. The disclosed methodology is suitable for a variety of NC systems such as semiconductor, metal oxide, and noble metal of different sizes and shapes. The method may, for example, be employed with luminescent quantum dots (QDs), since rendering the as-synthesized QDs water-soluble and biocompatible, while preserving the high luminescent brightness and maintaining colloidal and chemical stability, is of great importance for biological applications. The disclosed nucleotides- and/or nucleosides-capped QDs, after phase transfer, endow QDs with excellent fluorescent and colloidal stability independent on the pH and ionic strength, minimal hydrodynamic size, and are thus suitable potential fluorophores in biomedical imaging applications. The disclosed reversibly water-soluble NCs can be readily further functionalized by hydrophobic ligands via a secondary ligand-exchange reaction, allowing fully reversible phase transfer and surface functionalization of NCs. Also provided are methods of using the disclosed reversibly water-soluble nanocrystals to label a biological cell or molecular component of such cell.

### BRIEF DESCRIPTION OF THE DRAWINGS

FIGS. 1A-D—are transmission electron microscopy (TEM) images of initial oil-soluble NCs before phase transfer: (A) CdSe/CdS/ZnS QDs, (B) Bi₂S₃ nanorods, (C) MnO nano-cubes, and (D) Au nanadots.

FIGS. 2A-B—are luminescence images of initial oil-soluble QDs in hexane, and alcohol-soluble QDs dispersed in methanol and ethylene glycol (EG) after phase transfer with use of AMP lamination by room light (A) and UV light (B).

FIGS. 3A-D—are TEM images of AMP-modified NCs dispersed in water derived from the initial oil-soluble ones via phase transfer: (A) CdSe/CdS/ZnS QDs (6.0 nm), (B) Bi2S3 nanorods (8 nm×46 nm), (C) MnO nano-cubes (23 nm), and (D) Au nanodots (7.5 nm). The insets show photographs of the initial OAm-capped NCs dispersed in hexane (left) and the resulting AMP-capped NCs dispersed in water (right) after phase transfer.

FIGS. 4A-D—are luminescence images of initial oil-soluble QDs solutions in hexane, and water-soluble QDs in water under UV light irradiation after phase transfer with use of AMP (a) and adenosine (b) as phase transfer reagents, respectively. UV-vis (c) and PL (d, λₑₓ=360 nm) spectra of initial hydrophobic CdSe/CdS/ZnS QDs in hexane solutions (solid curves), and the corresponding AMP-capped QDs in aqueous media (dashed curves). Note: the PL intensities of all oil-soluble QDs were normalized to 100, and the PL spectra of the corresponding water-soluble QDs after phase transfer were recorded under the same condition as for oil-soluble QDs.

FIGS. 5A-B—are TEM images of initial oil-soluble QDs (A) and corresponding water-soluble ones after phase transfer with use of AMP (B). Inset: Dynamic light-scattering histograms of corresponding water-soluble QDs with average hydrodynamic diameter of 7.1 nm.

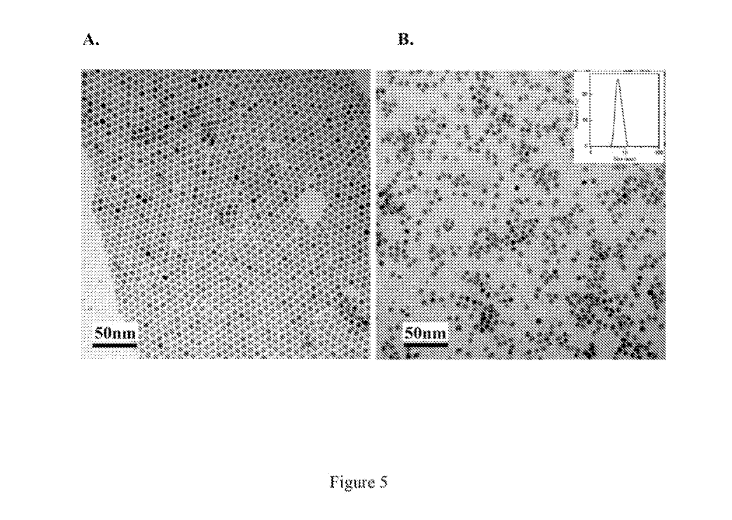

FIGS. 6A-C—are Fourier-transform infrared (FTIR) spectra of initial oil-soluble OAm-QDs (A), AMP-QDs after phase transfer (B), and free sodium Na₂-AMP (C).

FIG. 7—is an FTIR spectrum of the OAm-capped QDs recovered from the water-soluble AMP-QDs by phase-transfer back to the hydrophobic phase.

FIGS. 8A-C—are (A) a schematic illustration of the reversible phase transfer process of QDs sample dispersed in hexane and water. (B) Relative PL intensity variation of oil- and water-soluble QDs in each cycle of phase transfer process. (C) Representative PL emission spectra of the initial oil-soluble QDs (black line), water-soluble AMP-QDs after phase transfer (red line), and the recovered QDs (blue line).

FIGS. 9A-B—show luminescence stability of AMP-QDs in PBS buffers with different pH values. (A) Temporal evolution of relative PL intensities of AMP-QDs samples under different pH values. (B) Luminescence images of AMP-QDs samples with different pH values under UV light irradiation after stored for 30 days.

FIGS. 10A-B—show colloidal stability of AMP-QDs in NaCl solutions with different concentrations. (A) UV-vis spectra of AMP-QDs in NaCl solutions for one day, and (B) temporal evolution of relative PL intensities of AMP-QDs in NaCl solutions.

FIG. 11—is a graph showing temporal evolution of photo-luminscence (PL) intensity of AMP-capped QDs under the irradiation by a UV lamp at 254 nm excitation together with a reference sample MPA-QDs.

FIG. 12 is a graph showing temporal evolution of PL peak intensities of AMP-QDs and referenced MPA-QDs in the process of heating at 100° C. The first measurement point corresponds to samples at room temperature.

FIG. 13A-B—are representative images of HO-8910 cancer cells (A) and SMMC-7721 cancer cells (B) incubated with adenosine-QDs under different, incubation times.

### DETAILED DESCRIPTION

The ability to produce reversibly water-soluble and biocompatible nanocrystals (NCs) that preserve their detectible signal (e.g. high luminescent brightness), while maintaining colloidal and chemical stability, is of great importance for biological applications. Several strategies have been previously described for reversibly transferring NCs between hydrophobic and hydrophilic media. These include using weak surface-binding molecules, or using stimulus responsive ligands, such as electric field-, temperature-, or pH-sensitive ligands, and ligands that can undergo reversible host-guest complexation. Unfortunately, all reported reversible phase transfer approaches suffer from one or more of the following problems: i) the phase transfer process will deteriorate physicochemical properties of NCs, such as colloidal stability and optical properties; ii) the methods only work for certain NC systems; iii) NCs may be dispersed in either polar or nonpolar solvent, but not truly reversible between aqueous and organic media; iv) the phase transfer reagents require tedious synthesis and are expensive.

Presently disclosed is a general ligand-exchange methodology using nucleotides and/or nucleosides to replace the initial hydrophobic ligands (with long hydrocarbon tails) on the surface of NCs, thus rendering the initial oil-soluble NCs reversibly water-soluble. A reversibly water-soluble nanocrystal comprising a core nanocrystal capped by a plurality of nucleotides, a plurality of nucleosides, or a combination thereof is provided. The disclosed methodology is suitable for a variety of NC systems such as semiconductor, metal oxide, and to noble metal of different sizes and shapes. The method may, for example, be employed with luminescent quantum dots (QDs), since rendering the as-synthesized oil-soluble QDs water-soluble and biocompatible, while preserving the high luminescent brightness and maintaining colloidal and chemical stability, is of great importance for biological applications. The disclosed nucleotides- and/or nucleosides-capped QDs, after phase transfer, endow QDs with excellent fluorescent and colloidal stability independent on the pH and ionic strength, minimal hydrodynamic size, and are thus suitable potential fluorophores in biomedical imaging applications. The disclosed reversibly water-soluble NCs can be readily further functionalized by hydrophobic ligands via a secondary ligand-exchange reaction, allowing fully reversible phase transfer and surface functionalization of NCs.

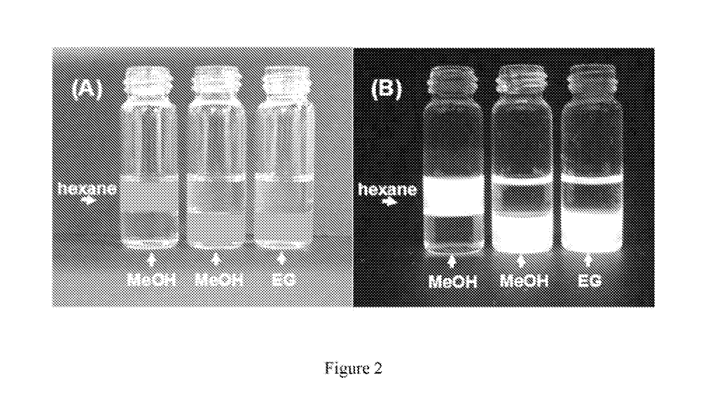

The NCs used were synthesized following standard protocols (as further described in the Examples) with use of capping ligands (mainly oleic acid and OAm) containing long hydrocarbon tails to render NCs stable and dispersible in hydrophobic media. For a typical ligands exchange procedure, a nucleotide or nucleoside solution in ethanol was added into the purified organics-capped NCs in a nonpolar solvent (such as hexane, chloroform). The two-phase mixture containing immiscible layers of ethanol and hexane was vigorously stirred for about 30 min, after which deionized water was added into the solution and the NCs was completely transferred from organic layer to aqueous phase. A series of nucleotide monophosphates (RNA monomer) including AMP, GMP, CMP and their corresponding nucleosides have been proven to possess this capability to render the initial organic ligands capped oil-soluble NCs water-soluble. Significantly, this phase transfer strategy can be successfully applied to NCs having a variety of compositions, sizes, and shapes, including semiconductor NCs such as CdSe/CdS/ZnS core/shell QDs (6.0 nm), Bi₂S₃ nanorods (8 nm×46 nm, FIG. 2B), metal oxides such as cube-shaped MnO nanostructures (23 nm, FIG. 2C), noble metal Au nanodots (7.5 nm, FIG. 2D), as a general avenue to obtain water-soluble NCs (see Figures). From the TEM images, it was found that size, morphology, or size distribution of the NCs has no observable alteration before and after phase transfer. In particular, the resulting nucleotides-capped NCs dispersions in water are stable for several months without any detectable aggregation or precipitation. Thus, this method provides a general and facile strategy for decorating hydrophobic NCs of various materials with hydrophilic ligands without changing their morphology and composition.

In one aspect, there is provided a reversibly water-soluble nanocrystal comprising a core nanocrystal capped by a plurality of nucleotides, a plurality of nucleosides or a combination thereof. The core nanocrystal may comprise a quantum dot, a nanorod, a metal is oxide nanocube, or a noble metal nanodot. The core nanocrystal may be a quantum dot having a core/shell/shell structure, such as CdSe/CdS/ZnS. The core nanocrystal may be a nanorod, such as Bi₂S₃. The core nanocrystal may be a nanocube, such as MnO, or may be a gold nanodot. Methods of synthesizing nanocrystals are well known in the art (see, e.g., Qu et al., J. Am, Chem. Soc. (2002) 124: 2049; Li et. al., J. Am. Chem. Soc. (2003) 125: 12567; Xie et al., J. Am. Chem. Soc. (2005) 127: 7480).

The plurality of nucleotides may comprise adenosine 5′-monophosphate, guanosine 5′-monophosphate, cytidine 5′-monophosphate, uridine 5′-monophosphate, thymidine 5′-monophosphate, or combinations thereof. The plurality of nucleosides may comprises adenosine, guanosine, cytidine, uridine, thymidine, inosine, or combinations thereof. Alternatively, the plurality of nucleotides may comprise adenosine 5′-diphosphate, guanosine 5′-diphosphate, cytidine 5′-diphosphate, uridine 5′-diphosphate, thymidine 5′-diphosphate, adenosine 5′-triphosphate, guanosine 5′-triphosphate, cytidine 5′-triphosphate, uridine 5′-triphosphate, thymidine 5′-triphosphate, or combinations thereof. The plurality of nucleotides may also alternatively comprise deoxynucelotides and the plurality of nucleosides may also alternatively comprise deoxynucleosides.

Nucleotide and nucleosides (including their mono-, di-, and tri-phosphate forms as well as deoxy- forms) are extensively well known, are commercially available from a variety of vendors (e.g. Sigma Chemical), and are cheap raw material for commercial use. They alternatively may be synthesized by methods well known in the art.

The disclosed reversibly water-soluble nanocrystal may be further characterized by being soluble and stable in water for at least about sixty days without substantial aggregation or precipitation. It may be further characterized by retaining substantially the same size, morphology, and size distribution when dispersed in water as compared to the core nanocrystal when capped by hydrophobic ligands and dispersed in hydrophobic media. It may be further characterized by retaining substantially the same luminescence brightness and colloidal stability when dispersed in water as when dispersed in a hydrophobic media.

The disclosed reversibly water-soluble nanocrystal may be further characterized in that the plurality of nucleotides, the plurality of nucleosides or the combination thereof that have capped the core nanocrystal form a layer that is equal to or less than about five nanometers thick. It may be further characterized in that the nanocrystal does not comprise any hydrophobic ligands. It may be still further characterized by being soluble in a water-miscible alcohol. The alcohol may be methanol, ethanol, ethylene glycol, or combinations thereof.

The reversibly water-soluble nanocrystal may be further characterized in that the water-solubility may be reversed by displacing the plurality of nucleotides and/or nucleotides that have capped the core nanocrystal with a plurality of hydrophobic capping ligands. Such reversal of the water-solubility may be accomplished at room temperature in one hour or less. The conversion between water-solubility and water-insolubility may be carried out for at least ten cycles without significantly altering the performance characteristics of the nanocrystal.

The reversibly water-soluble nanocrystal may further be characterized by retaining substantially the same luminescence brightness and colloidal stability when dispersed in aqueous media having a pH in the range of three to thirteen as when dispersed in aqueous media of neutral pH. It may further be characterized by retaining substantially the same luminescence brightness and colloidal stability when dispersed in aqueous media having a salt concentration in the range of one molar to five molar as when dispersed in aqueous media having a salt concentration of zero molar.

The reversibly water-soluble nanocrystal may be further characterized by having superior resistance to photo-oxidation-induced precipitation in aqueous media when compared to a similar nanocrystal capped with mercaptopropionic acid in the same aqueous media. It may further be characterized by the core nanocrystal being a quantum dot, and wherein the superior resistance to photo-oxidation-induced precipitation is measured as retaining to substantially the same luminescence brightness after one hour exposure to 6-watt, 254 nm UV irradiation as prior to such exposure.

The reversibly water-soluble nanocrystal may further be characterized by having superior resistance to temperature-induced precipitation in aqueous media when compared to a similar nanocrystal capped with mercaptopropionic acid in the same aqueous media.

It may further be characterized by the core nanocrystal being a quantum dot, and wherein the superior resistance to temperature-induced precipitation is measured as retaining substantially the same luminescence brightness after one-half hour at one hundred degrees Celsius as when at room temperature.

The disclosed reversibly water-soluble nanocrystal may be further characterized by retaining substantially the same luminescence brightness and colloidal stability within a biological cell when compared to outside a cell in aqueous media.

In another aspect, there is provided a nanocrystal composition comprising a plurality of reversibly water-soluble, nucleotide- and/or nucleoside-capped nanocrystals, wherein the nanocrystals:

(i) are soluble and stable in water for at least two months without any substantial aggregation or precipitation;

(ii) retain substantially the same size, morphology, size distribution, and performance characteristics when dispersed in water as compared to a similar nanocrystal capped by hydrophobic ligands and dispersed in hydrophobic media; and

(iii) may be rendered reversibly water-insoluble by displacing the nucleotides and/or nucleosides that cap the nanocrystals with a plurality of hydrophobic capping ligands.

In another aspect, there is provided a method for producing reversibly water-soluble nanocrystals, the method comprising the steps of:

(i) providing water insoluble nanocrystals comprising core nanocrystals capped by a plurality of initial hydrophobic ligands; and

(ii) contacting the water insoluble nanocrystals of step (i) with a plurality of nucleotides, a plurality of nucleosides, or a combination thereof, thereby replacing the initial hydrophobic ligands that cap the core nanocrystal with a cap of nucleotides, nucleosides, or a combination thereof, thus rendering the core nanocrystal reversibly water-soluble.

In another aspect, there is provided a method for reversibly transferring nanocrystals between a hydrophobic solution and a hydrophilic solution, the method comprising:

(i) providing a hydrophobic solution comprising core nanocrystals capped by a plurality of hydrophobic ligands;

(ii) mixing the hydrophobic solution with a solution comprising a plurality of nucleotides and/or nucleosides in order to replace the hydrophobic ligands capping the core nanocrystal with the nucleotides and/or nucleosides, thereby producing a ligand-exchanged solution comprising nucleotide- and/or nucleoside-capped nanocrystals; and

(iii) mixing the ligand-exchanged solution with a hydrophilic solution, whereby the nucleotide- and/or nucleoside-capped nanocrystals transfer from the hydrophobic, ligand-exchanged solution to the hydrophilic solution.

In the above-described method, the hydrophobic solution may be hexane. The hydrophilic solution is water, a water-miscible alcohol, or a combination thereof. The method may further comprise the step of:

(iv) mixing the hydrophilic solution with (a) a solution comprising a plurality of hydrophobic ligands and (b) a secondary hydrophobic solution in order to replace the nucleotides and/or nucleosides capping the core nanocrystal with the hydrophobic ligands, thereby producing a secondary ligand-exchanged solution comprising hydrophobic ligand-capped nanocrystals, whereby the hydrophobic ligand-capped nanocrystals transfer from the hydrophilic solution to the secondary hydrophobic solution.

The hydrophobic ligands may comprise organic amine ligands, for example oleylamine, hexadecylamine, dodecylamine, or combinations thereof. The secondary hydrophobic solution may be hexane. Organic amine ligands suitable for capping nanocrystals in hydrophilic media are well known in the art. Similarly, hydrophobic solutions suitable for conducting extraction of nanocrystals between hydrophobic and hydrophilic phases are well known in the art.

In another aspect, there is provided a composition comprising a biological reagent coupled to a reversibly water-soluble nanocrystal comprising a core nanocrystal capped by a plurality of nucleotides, a plurality of nucleosides, or a combination thereof. The biological reagent may comprise an antibody, a nucleic acid probe, an enzyme substrate, a binding protein, or combinations thereof. Such reagents are widely commercially available from a number of vendors, and methods for generating such reagents are well known in the art. For example, the antibody may specific for phosphorylated epidermal growth factor receptor (EGFR), and the disclosed composition thus allows for the nanocrystal-based detection of the binding of such antibody to phosphorylated EGFR. By way of a second example, the nucleic acid probe may be specific for a gene translocation mutation, such as the BCR-ABL translocation in leukemia, and the disclosed composition thus allows for the nanocrystal-based detection of the binding of such probe to BCR-ABL.

In another aspect, there is provided a method for labeling a biological cell or molecular component of such cell, the method comprising the step of:

contacting the cell or molecular component with a reversibly water-soluble nanocrystal comprising a core nanocrystal capped by a plurality of nucleotides, a plurality of nucleosides, or a combination thereof.

In another aspect, there is provided a method for labeling a biological cell or molecular component of such cell, the method comprising the step:

contacting the cell or molecular component with a composition comprising a biological reagent coupled to a reversibly water-soluble nanocrystal comprising a core nanocrystal capped by a plurality of nucleotides, a plurality of nucleosides, or a combination thereof.

In another aspect, there is provided a biological cell or molecular component of such cell labeled with a reversibly water-soluble nanocrystal comprising a core nanocrystal capped by a plurality of nucleotides, a plurality of nucleosides, or a combination thereof. The biological cell or molecular component of such cell may be a cancer cell, or other mammalian cell of interest, such as a neural cell. The molecular component of such cell may be a nuclear membrane, or other organelle or cell structure, such as mitochondria.

In another aspect, there is provided a biological cell or molecular component of such cell labeled with a composition comprising a biological reagent coupled to a reversibly water-soluble nanocrystal comprising a core nanocrystal capped by a plurality of nucleotides, a plurality of nucleosides, or a combination thereof.

The following Examples are provided only to further illustrate the disclosure, and are not intended to limit its scope, except as provided in the claims appended hereto. The present disclosure encompasses modifications and variations of the methods taught herein which would be obvious to one of ordinary skill in the art.

### EXAMPLE

### Synthesis of Oil-Soluble Nanocrystals

In these Examples, adenosine 5′-monophosphate (AMP), adenosine, adenine, guanosine 5′-monophosphate (GNP), guanosine, cytidine 5′-monophosphate (CMP), and cytidine were purchased from Sigma. (MPA, >99%), tetramethylammonium hydroxide pentahydrate (TMAH, >95%), oleylamine (OAm, 70%), 1-hexadecylamine (98%), 1-dodecylamine (>99%), oleic acid (99%), 1-octadecene (ODE, >95%) were obtained from Aldrich and used without further purification. All organic solvents such as hexane, dichloromethane, ethanol, methanol were of analytical grade and obtained from commercial sources and used as received. Deionized water was used throughout.

A. CdSc/CdS/ZnS Core/Shell/Shell Quantum Dots (QDs).

CdSe core nanocrystals were prepared via a modified literature method (see, e.g., Qu et al., J. Am. Chem. Soc. (2002), 124: 2049. Typically, 25.6 mg (0.2 mmol) of CdO, 1.2 g of TOPO, 1.0 ml of oleic acid and 4.0 ml of ODE were loaded in a 50 mL three-neck flask clamped in a heating mantle. The mixture was heated to 320-330° C. under argon flow and resulted in a colorless clear solution, which was then cooled to 315° C. At this temperature, 2.4 mL of the Sc precursor solution, which was made by dissolving selenium (79.0 mg) in TOP (4.0 ml) and ODE (6.0 ml) by sonication, was quickly injected into the reaction flask. After the injection, the reaction temperature was set at ˜270 ° C. for the growth of the nanocrystals with different periods of time (10 s-3 min) to get nanocrystals with desired size. The reaction mixture was then allowed to cooled to ˜60° C. and 10.0 ml of hexane/CH₃OH (v/v, 1:1) was used as the extraction solvent to separate the nanocrystals from byproducts and unreacted precursors. The as-prepared CdSe solution was further purification by centrifugation and decantation with the addition of acetone and then the CdSe NPs were redispersed in hexane.

Stock solutions preparation. The Zn precursor solution (0.1 M) was prepared by dissolving 219.5 mg (1 mmol) Zn(OAc)₂.2H₂O in 10.0 mL ODE at 160° C. The sulfur precursor solution (0.1 M) was obtained by dissolving sulfur in ODE at 120° C. The Cd precursor solution (0.1 M) was prepared by dissolving 128.4 mg (1 mmol) CdO in 2.0 ml oleic acid and 8.0 mL ODE at 160° C. Each stock solution was stored at room temperature. Synthesis of CdSe/CdS/ZnS core/Shell/Shell QDs. The successive ion layer adsorption and reaction (SILAR) technique was adopted for the growth of CdSe/CdS/ZnS core/shell/shell nanocrystals (see, e.g. Li et al., J. Am. Chem. Soc. (2003), 125:12567; Xie et al., J. Am. Chem. Soc. (2005), 127: 7480. In a typical procedure, a chloroform solution of purified 3.5 nm CdSe QDs containing 0.1 mmol of CdSe, 1.0 mL of oleylamine and 4.0 mL of ODE were loaded in a 50 mL flask. The flask was then pumped down at room temperature for 20 min to remove the chloroform and at 100° C. for another 20 min while flushing the reaction system twice with a flow of argon. Subsequently, the reaction mixture was further heated to 230° C. for the overgrowth of the CdS shell. The Cd precursor stock solution was added into the reaction flask, after 10 min when the Cd precursor was fully deposited around the CdSe surface, an equimolar amount of S precursor stock solution was added into the reaction system. When the first monolayer of CdS was deposited around the CdSe cores, another Cd/S precursor solution was added alternately at approximately 10 min intervals. The volume of the precursor stock solution added in each cycle was the amount needed for a whole monolayer of CdS shell. The amount was calculated from the respective volumes of concentric spherical shells with 0.35 nm thickness for one monolayer (ML) of CdS (e.g. 0.7, 1.0, 1.3 mL for the 1^{st} , 2^{nd}, and 3^{rd} ML, respectively). Then the reaction temperature was set at 200° C. for the overgrowth of ZnS shell. The Zn/S precursor stock solution was added into the reaction flask at intervals of 20 min. To monitor the reaction, aliquots were taken before a new cycle of injection and their corresponding UV-vis and PL spectra were recorded. The reaction was terminated by allowing the reaction mixture to cool down to room temperature. The purification procedure was similar to that for CdSe core nanocrystals.

B. Synthesis of Bi₂S₃ Nanorods.

Nanorods were produced essentially according to standard methods (see, e.g. Wu, et al., Nano Res. (2010), 3: 379-386). 31.5 mg (0.1 mmol) of BiCl3 powder was added to a flask containing 2.0 mL of OAm followed by degassing at 70° C. for 5 min under vacuum to remove the moisture and oxygen. The reaction vessel was then filled with nitrogen and the temperature was increased to 150° C. with a heating rate of 10° C./min and the vessel maintained at this temperature until the complete dissolution of BiCl3 powder to give a white milky solution. Then 2.0 mL of OAm containing 11.3 mg (0.15 mmol) of thioacetamide was injected into the reaction system. After the addition of thioacetamide, the temperature of the reaction system was further increased to 180° C. and the vessel maintained at this temperature for 5-10 min. The reaction mixture was cooled to room temperature and the resulting nanostructures were precipitated by anhydrous ethanol, The Bi2S3 NPs were redispersed in hexane to give a brown dispersion.

C. Synthesis of MnO Nanocubes

Nanocubes were produced essentially according to standard methods (see, e.g., Zhong et al., J. Phys. Chem. B (2006), 110: 2-4; Yin et al., J. Am. Chem. Soc. (2003), 125: 10180-10181). 24.5 mg (0.1 mmol) of Mn(OAc)2 and 2 mL of oleylamine were mixed with 5 mL ODE in a 50 mL flask. Under nitrogen flow, the mixture was quickly heated to 130° C. under magnetic stirring. The formed solution was kept at this temperature for 10 min and cooled down to room temperature. Then 30 mL of anhydrous ethanol was added into the solution, and the suspension was centrifuged at 4000 rpm for 5 min. The supernatant was discarded and the MnO NPs were redispersed in hexane to give a brown dispersion.

D. Synthesis of Au Nanodots.

Gold nanodots were produced essentially accordingly to standard methods (see, e.g., Shen et al., Chem. Mater, (2008), 20: 6939-6944). 34.0 mg (0.1 mmol) of HAuCl₄3H₂O and 1.0 mL of oleylamine were mixed with 15 mL toluene in a 50 mL flask. Under nitrogen flow, the mixture was slowly heated to 65° C. under magnetic stirring. The formed solution was kept at this temperature for 6 h and cooled down to room temperature. Then 30 mL of anhydrous ethanol was added into the solution, and the suspension was centrifuged at 4000 rpm for 5 min. The supernatant was discarded and the Au NPs were redispersed in hexane to give a red dispersion.

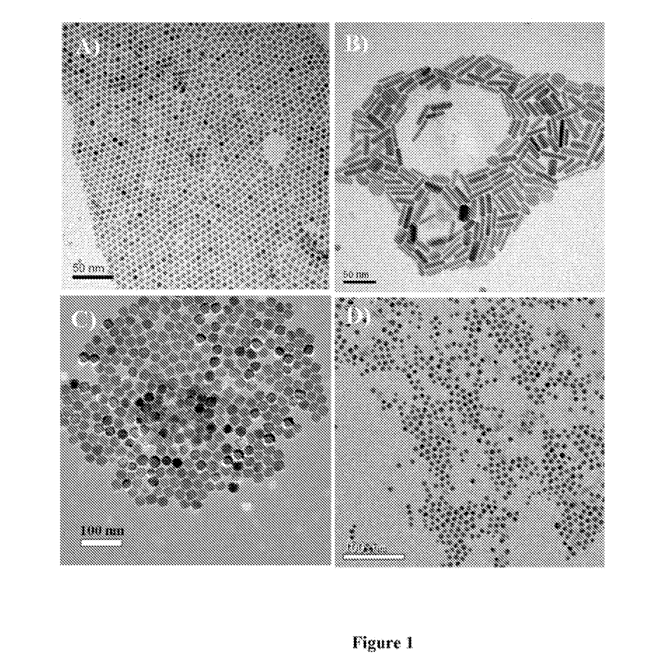

FIG. 1 shows TEM images of initial oil-soluble NCs before phase transfer: (A) CdSe/CdS/ZnS QDs, (B) Bi₂S₃ nanorods, (C) MnO nano-cubes, and (D) Au nanodots. Typical morphology, size, and dispersion of each type of exemplary water-insoluble nanocrystal is shown.

### EXAMPLE 2

Production of Water-Soluble Nanocrystals

Exchange of the native hydrophobic ligands on QDs surface (as produced in Example 1A) by AMP (or other nucleotide or nucleoside ligands including adenosine, adenine, GMP, guanosine, CMP, and cytidine) was performed as follows. Typically, 1.0 g of AMP was dissolved in 3.0 mL of ethanol and the pH of the resulting solution was adjusted to 10 with the use of concentrated NaOH or TMAH solution. Then 0.3 mL of the obtained AMP solution in ethanol was added dropwise into a purified QDs solution in hexane (or CHCl₃) (10⁻⁶ M, 20.0 mL), and vigorously stirred for 30 min. Subsequently, deionized water was added into the solution. The QDs were found to be successfully transferred from the hexane phase on the top to the water phase in the bottom. The colorless organic phase was discarded and the aqueous phase containing the QDs was collected. The excess amount of free ligand was removed by centrifugation purification with use of acetone. The supernatant was discarded and the pellet was then re-dissolved in water and repeated this centrifugation-decantation process three times to get the purified QDs aqueous solutions.

FIG. 2 shows luminescence images of initial oil-soluble QDs in hexane, and alcohol-soluble QDs dispersed in methanol and ethylene glycol (EG) after phase transfer with use of AMP lamination by room light (A) and UV light (B). The images show that phase-transfer of nucleotide/nucleoside-capped core nanocrystals has occurred, and these capped nanocrystals are dispersed in the aqueous (water-miscible MeOH) phase.

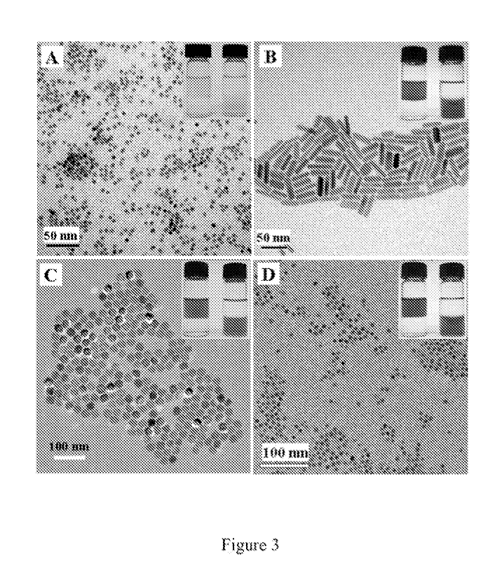

FIG. 3 shows TEM images AMP-modified NCs dispersed in water derived from the initial oil-soluble ones via phase transfer; (A) CdSe/CdS/ZnS QDs (6.0 nm), (B) Bi₂S₃ nanorods (8 nm×46 nm), (C) MnO nano-cubes (23 nm), and (D) Au nanodots (7.5 nm). The insets show photographs of the initial OAm-capped NCs dispersed in hexane (left) and the resulting AMP-capped NCs dispersed in water (right) after phase transfer. The images indicate that nucleotide/nucleoside-capped, water-soluble nanocrystals maintain essentially the same size, morphology, and distribution in aqueous phase as the initial, equivalent hydrophobic ligand-capped core nanocrystals in oil phase.

In these Examples, UV-vis and PL spectra were obtained on a Shimadzu UV-2450 spectrophotometer and a Cary Eclipse (Varian) fluorescence spectrophotometer, respectively. FT-IR spectra were measured with samples pressed into a KBr plate and recorded from 4000 to 400 cm⁻¹ using a Nicolet Magna-IR 550 FT-IR spectrometer. Transmission electron microscopy (TEM) images were taken on a JEOL JEM-1400 at an acceleration voltage of 100 kV. Dynamic light scattering (DLS) was conducted with a Zeta Sizer nano series laser light scattering system (Malvern Instrument Corporation). Confocal fluorescence imaging was performed with an OLYMPUS ZX81 laser scanning microscopy and a 60× oil-immersion objective lens.

### EXAMPLE 3

### Characteristics of Nucleotide/Nucleoside-Capped Nanocrystals

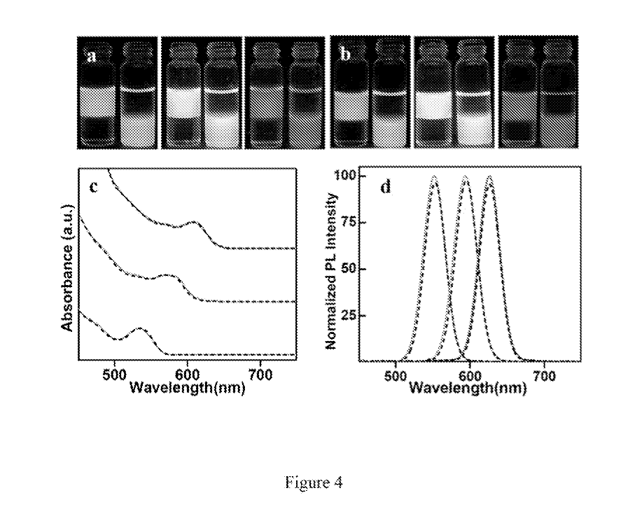

The phase transfer experiments of CdSe/CdS/ZnS core/shell QDs were performed on three typical emission wavelengths of 552, 594, and 626 nm. The selected nucleotide monophosphates and nucleosides all showed superior performance for the phase transfer of the organic ligands capped hydrophobic QDs into water-soluble ones. As a demonstration, FIG. 4 shows the luminescence images of three QDs samples before and after phase transfer with use of AMP and adenosine, respectively. No distinguishable luminescence brightness of the QDs before and after phase transfer with use of either AMP or adenosine, demonstrating the superior performance of both AMP and adenosine in phase transfer of oil-soluble QDs. The phase transfer process was facile and occurred with nearly 100% efficiency, which was determined by observing almost no luminescence emission and no absorption corresponding to QDs in the optical spectra of the organic phase after the phase transfer.

After phase transfer, the AMP-capped QDs aqueous solutions exhibited identical absorption and PL emission spectral profiles to those of original hydrophobic QDs in hexane (see FIG. 3). This indicates that the surface ligands have no effects on the electronic properties of the inorganic QDs cores and no aggregation or surface degradation of the QDs occurred upon the process of phase transfer. The original high florescent brightness was preserved for all selected QDs samples with different emission colors after the phase transfer and the water-soluble AMP-capped QDs show almost identical PL QYs in the range of 50-65% as those of the original oil-soluble ones. It should be noted that heavy loss of luminescence brightness of QDs after phase transfer into aqueous solutions was commonly observed in previously reported attempts to generate water-soluble nanocrystals.

FIG. 4 shows TEM images of 6.0 nm QDs with emission wavelength of 594 nm before and after phase transfer with use of AMP as phase transfer reagent, respectively. TEM measurements showed that the AMP-capped QDs provided well-dispersed distributions of nanoparticles. They were well isolated from each other and no aggregation or clumping occurred, revealing that the NCs size and shape are preserved. The aggregate-free nature of the QDs dispersions in water is further verified by the monomodal size distributions measured by DLS as shown in the inset of FIG. 4B. The average hydrodynamic diameter (HD) of AMP-QDs extracted from the DLS measurement results was about 7.1 nm, which is only a little larger than the size observed by TEM (6.0 nm). The thickness of the capping layer AMP is deduced as only about 1.1 nm. This compact shell matches the multi-dentate coordination mode of AMP ligand on the QDs surface as described below. The observed HD of these AMP-capped QDs is remarkably smaller than those of amphiphilic polymer coated QDs, which have HDs on the level of 20-50 nm. The reduced HD of water-soluble luminescent QDs is important for cell labeling and single-particle tracking applications. These above features suggest that nucleotides ligands benefit the surface stabilization of QDs for their high fluorescence QYs, well monodispersibility, and remarkably small HD.

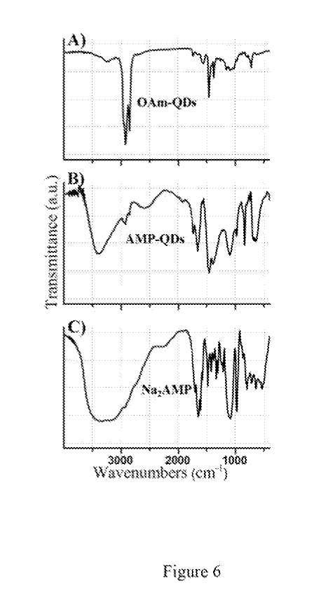

The fact that phase transfer was achieved via the ligand-exchange was confirmed by FTIR spectroscopy. The FTIR spectra of the initial OAm-capped QDs (FIG. 6) show the characteristic C—H stretching vibrations, derived from long hydrocarbon chain portion in OAm molecules, at 2800-3000 cm⁻¹ with strong intensity; while those of water-soluble QDs show almost no signals in the C—H stretching vibration region. This indicates that the original OAm molecules attached to the QDs surface were near completely removed after phase transfer. It was found that the spectra of water-soluble QDs after phase transfer (FIG. 6B) show a similar spectral profile to those of free Na₂-AMP (FIG. 6C). Thus it can be concluded that the capping ligand on QDs surface has been changed to AMP after this phase transfer. In the spectrum for AMP-QDs, the broad band around 3500 cm⁻¹ is assigned to the O—H vibration of solvated water molecules and the OH group from the sugar portion of AMP. The presence of solvated water molecules is consistent with the hydrophilic nature of the AMP-QDs. Due to the coordination of AMP molecules on the surface of QDs, most of the sharp vibration in the spectra of free AMP molecules are broadened in those of AMP-QDs and the broadening phenomenon for capped ligands on surface of nanoparticles has been commonly observed in previous literature reports. The features associated with the vibration of phosphate group (cm 950-1100 cm⁻¹) and the purine ring (ca. 1450-1650 cm⁻¹) of the nucleotides were all broadened in the AMP-QDs. The FTIR spectroscopy analysis confirms that the initial OAm capping molecules are indeed replaced by AMP from the surface of QDs in the process of phase transfer.

The facts that adenosine molecule without the existence of phosphate group can also render the initial oil-soluble QDs water-soluble via ligand exchange can deduce that the purine group of AMP or adenosine is evolved into the coordination with the surface metal atoms of QDs. Furthermore, the small molecule adenine is a model compound to perform the phase transfer. Similarly, adenine can also render the initial oil-soluble OAm-QDs water-soluble as done by AMP or adenosine. This further confirms that purine ring indeed coordinate to the nanoparticles.

The disclosed nucleotides- and/or nucleosides-capped QDs obtained through phase transfer can disperse in water-soluble alcohols such as methanol, ethanol, and ethylene glycol (EG). Images of emissions color of initial oil-soluble QDs dispersed hexanes and AMP-capped QDs dispersed in MeOH and EC after ligand exchange are shown in FIG. 2. Since ethanol and hexane are partly miscible and no clear boundary can be formed, the photograph of QDs dispersed in ethanol was not given. Similar to the case of QDs dispersed in aqueous media, the MPA-QDs dispersed in alcohol media also preserve the high fluorescent brightness and spectral profile. It should be noted that the access of high quality (high PL QY and high colloidal stability) alcohol-dispersible QDs is even more difficult than preparation of water-soluble luminescent QDs and thus few tenable approaches have previously been reported. In addition, the access of alcohol-soluble NCs plays the crucial role for the silica coating process via stober route. It is not unexpected for the alcohol-soluble of the nucleotides capped QDs since multiple hydroxyl groups existing in the nucleotide molecules.

### EXAMPLE 4

### Reversible Phase Transfer of Water-Soluble Nanocrystals

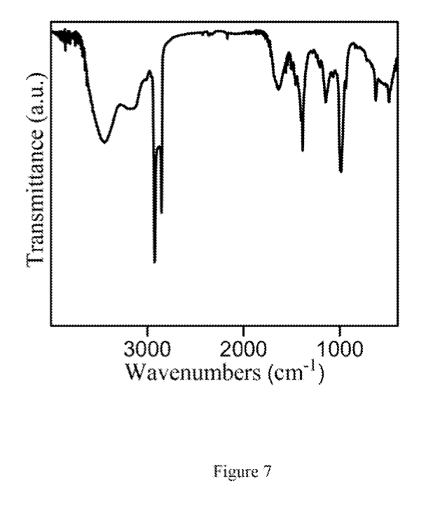

A significant benefit of the phase transfer method disclosed herein is that the resulting water- and alcohol-soluble nucleotides- and/or nucleotides-modified nanocrystals can be further transferred back to oil-soluble with use of hydrophobic ligands. This allows for reversible phase transfer and further surface functionalization of NCs. Upon the addition of organic amine ligands such as OAm, hexadecylamie, or dodecylamine to the nucleotides-capped QDs dispersion in water or alcohol combined with hexane, QDs were found to transfer completely from the water or alcohol layer to the hexane layer after stirring for about 10 minutes, suggesting that QDs are successfully capped by hydrophobic amine ligands. The FTIR spectrum of the OAm-capped QDs recovered from the water-soluble AMP-Ws are shown in FIG. 7, which displays the characteristic C—H stretching vibration signals in the range of 2800-3000 cm⁻¹.

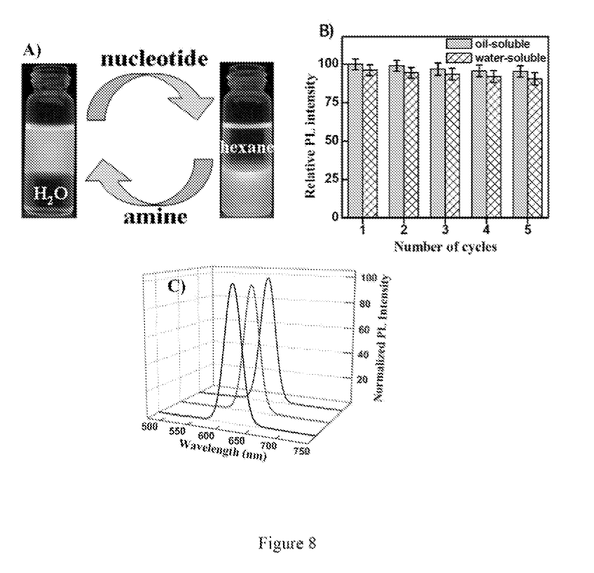

After transfer back to oil-phase, the hydrophobic QDs where purified by centrifugation to remove excess amount of free amine ligands, and again, adding nucleotide ligands to the recovered QDs solution in hexane following the previous phase transfer step, QDs will be rendered to water-soluble again. This completely reversible process can be performed at room temperature, providing a feasible method to obtain hydrophobic nanoparticles from aqueous medium, or vice versa. FIG. 8A illustrates the reversible phase transfer results performed on CdSe/CdSe/ZnS QDs. When oil-soluble QDs were transferred into water-soluble ones, the decrease of PL brightness is less than 5%; while the slightly decreased PL brightness can be recovered almost completely after transferred back into oil-soluble. It should be highlighted that the transfer between aqueous and oil phases can be repeated for more than 10 cycles without significantly altering the fluorescent brightness. FIG. 8B shows the variation of relative PL intensities of oil- and water-soluble QDs in each phase transfer cycle and the representative PL emission spectra are shown in FIG. 8C. This facilely reversible surface modification is attributed to the weak binding affinity of hydroxyl portion in nucleotides/nucleosides to the NC surface.

### EXAMPLE 5

### Stability Water-Soluble Nanocrystals

A. pH Stability,

To test pH stability of the water-soluble nanocrystals, 100 μL of purified concentrated AMP-QDs aqueous solution was added and mixed well into a 4.9 mL of PBS buffer solution with different pH values (different pH values were obtained with the addition of HCl or NaOH solution to the 50 mM phosphate buffer with initial pH of 7.0). The obtained AMP-QD solutions with various pH values were sealed and stored in the stark and monitored their PL spectra over time. By this procedure, the QDs solutions with different pH values were of identical concentration in the testing period. Since the PL spectra of all samples were measured under identical instrumental conditions, the PL intensities of the corresponding QDs can represent their corresponding QYs and can be used to compare their pH sensitivity.

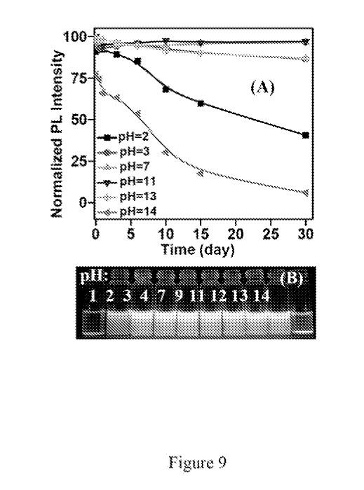

In FIG. 9A, the temporal evolution of PL intensities of AMP-capped QDs in representative pH buffer solutions were presented. As expected, the obtained AMP-coated QDs can remain high PL QYs and colloidal stability, aggregation-free over extended periods of time (months) in a broad pH range (3-13). It was observed that in the pH range of 3-13, highly fluorescent brightness can be preserved without significant luminescence loss (<10%) in a time period of 30 days. In both the extremely low pH of 2 and extremely high pH of 14, the PL intensity decreased in a relative fast fashion. After 15 days, the corresponding PL intensity can only retain 60% and 19%, respectively. The emission colors of AMP-capped QDs after staying for 30 days were shown in FIG. 9B. Except the samples at pH 1 or 14, all the samples showed bright luminescence without observed precipitation. This reflects highly stable property of their luminescence and superior dispersibility in a wide pH range. In comparison, thiol-functionalized carboxylic acid (such as MPA, DHLA) capped QDs can only keep high PL QY and colloidal stability at neutral to weak basic solutions as reported in previous literature, since a high pH value is favor for deprotonation of carboxylic acids and deprotonation of carboxyl groups is known to be the crucial factor to solubilize these carboxylated ligand coated QDs. This narrow pH range limits their potential biomedical application.

The superior colloidal and luminescent stability of the disclosed nucleotides/nucleosides-capped QDs may stem from the intrinsic molecular structure of nucleotides as proven in the nucleotides capped CdS QDs synthesized directly in aqueous media. As an instance, AMP is a compound with three hydrophilic groups, a phosphate group, two hydroxyl groups, and endocyclic and exocylic amine groups. Phosphoric acid is a strong acid with high pk_{α} value which is beneficial for the solubility in strong acidic environment and hydroxyl groups are non-ionic which is not sensitive to pH values and amine group is prone to protonation at acidic condition. For the same reason, our experimental results indicated that adenosine-capped QDs is also stable a pH 3-13. The colloidal and luminescent stability of nucleotides-QDs in acidic media suggests that they may be good candidates as intracellular imaging probes for QD applications because most intracellular organelles such as endosomes and lysosomes are acidic (pH 4-6).

B. Salt Stability.

The use of QDs in any sensing scheme requires that they exhibit long-term stability in solutions that span a wide range of electrolyte concentrations. The aggregation of QDs at elevated salt concentration may define the limitations of some biological applications in high ionic strength media, such as intracellular and in vivo studies, where the ionic concentration is known to be high. To test the colloidal stability of the nucleotides-QDs in electrolyte solutions, purified concentrated AMP-QDs solution was diluted into NaCl solutions with a series of different concentrations.

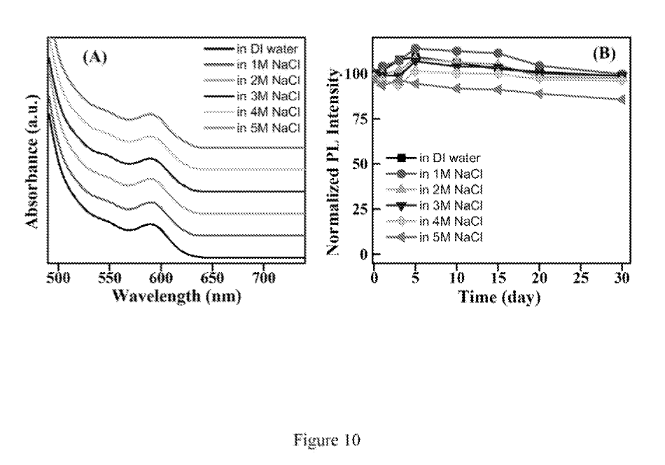

FIG. 10A shows the UV-vis absorption spectra of AMP-QDs incubated in NaCl solution with different concentrations for a whole day. For all the absorption spectra, up to the nearly saturated NaCl solution media (5.0 M), the baselines were all horizontal and no absorption tail at the long wavelength side was observed. This indicates that scatter light forum the colloidal dispersions did not exist and thus no aggregation occurred for the QDs dispersions throughout the whole NaCl concentration range. It is noted that the AMP-coated QDs solutions with different NaCl concentrations could still keep homogeneously dispersible after stored for more than 30 days. The instant PL spectra of AMP-coated QDs dispersed in different concentrations NaCl solutions kept a similar profile but with slightly variation of PL intensities (less than 3%) as shown in the first measurement points in each curves of FIG. 10B. During a period of 30 days, the PL intensity of all samples did not show significant quenching although some fluctuation was observed within 10% relative intensity variation. It is highlighted that the PL QYs have a 10-15% increase in the first 5-10 days when the QDs dispersed in solutions containing 0-2.0 M NaCl. The increase of PL intensity may due to the light irradiation effect during the storage stage. Multiple hydroxyl groups provide AMP-QDs a stable hydration layer, which is little affected by charged ions. This may give the reason for the superficial colloidal stability of AMP-QDs under high salinity condition. It is noteworthy that the extraordinary stability of disclosed AMP-stabilized QDs in high concentration salt solutions can be comparable to that of reported QDs functionalized by Tween derivatives, or zwitterionic ligands. This feature of high salinity tolerance is of special interest to expand their applications to biology and biomedicine.

C. Photo-Oxidation Stability.

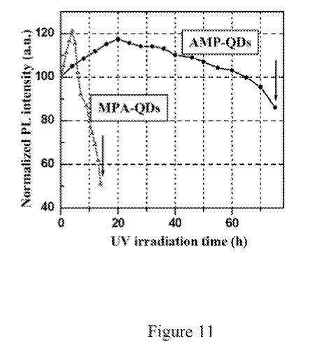

To investigate the photostability of the AMP-QDs in PBS buffer solution (pH=7), the MPA-capped QDs derived from the same batch of oil-soluble QDs were used as a reference and irradiated together with the AMP-QDs by a 6-watt UV light at 254 nm in the presence of oxygen under identical conditions. The selection of MPA-capped QDs as a reference is because MPA-QDs are the most common water-soluble ones derived from phase transfer via ligand exchange procedure and the obtained MPA-QDs exhibit one of the best results for water-soluble QDs. The used MPA-QDs were prepared strictly according to the modified procedure developed by Lee (see Pong et al., Langmuir (2008), 24: 5270-5276). The temporal evolution of relative PL intensity of both samples is summarized in FIG. 11, with initial PL intensity for each sample normalized to 100. It was found that shortly after the onset of irradiation, both QD samples increased in PL intensity (by a factor of 17-21%). This photo-enhancement effect was commonly observed in previous reports and the reason may be due to a surface rearrangement of ligands and/or photocatalytic annealing of surface atoms to repair defects and recombination centers that formed during a disordered ligand exchange. The PL enhancement effect by UV irradiation is much more instant for MPA-QDs than that for AMP-coated QDs. After approaching maximum value (4 h for MPA-QDs, and 20 h for AMP-QDs), the PL intensities in both samples decreased gradually. Within 15 h of UV light exposure, MPA-QDs precipitated gradually, which was in accordance with previous reports of photooxidation of the thiol-containing MPA ligand, resulting, in colloidal instability and aggregation. The facile oxidization feature of thiol group in MPA ligands deteriorate the photo-stability of the resulting QDs. In comparison, AMP-capped QDs also endured photo-bleaching but at a much slower rate than MPA-coated QDs. The bright luminescence of the AMP-QDs can be retained up to 60 h, after which the PL intensity decreased gradually and the QDs precipitated after irradiation of 75 h. This superior photo-stability of AMP-QDs may be attributed to the anti-oxidation capability of nucleotide ligands

D. Thermal Stability.

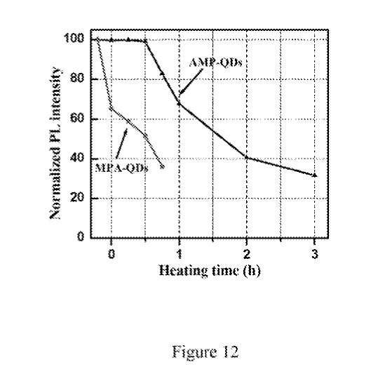

Thermal stability of the disclosed water-soluble, nucleotide/nucleotide-capped nanocrystals was evaluated by monitoring the evolution of relative luminescence intensity of both AMP-QDs and reference sample MPA-QDs in PBS buffer (pH=7) at 100° C. in nitrogen atmosphere. The purified AMP-QDs and MPA-QDs were loaded in a closed container and heated from room temperature to 100° C. in a period of 10 min and kept at this temperature for a certain period to monitor the variation of luminescence intensity. Timing started when the temperature reached 100° C. Experimental results showed that our AMP-QDs exhibited excellent thermal stability in boiling water. As shown in FIG. 12 when the samples were heated to 100° C. from room temperature, the corresponding PL intensities of AMP-QDs could preserve their high PL QY at room temperature and maintain this high brightness for 0.5 h, after then the PL intensity decrease gently and the PL intensity can retain ca. 70, 43 and 36% of the initial value at room temperature with heating time of 1, 2, and 3 h, respectively. Furthermore, the AMP-QDs could still keep homogenously dispersible at solution with the heating time up to 3 hours. While in the reference sample MPA-capped QDs, the PL intensity decreased much more sharply. When the temperature was raised from mom temperature to 100° C. (corresponding to time of 0 min), the PL intensity dropped about 40%. With extending heating, the MPA-QDs aggregated and precipitated gradually in a period of 1 h. These findings suggest that the exemplary, disclosed water-soluble nanocrystals (AMP-QDs) are much superior to MPA-QDs in the aspect of thermal stability.

### EXAMPLE 6

### Cell Labeling with Water-Soluble Nanocrystals

The nucleotides- and nucleosides-capped QDs are all very stable in cell culture medium without luminescence change or particle aggregation. To establish the suitability of the disclosed nanocrystals for biological applications, we examined the cellular uptake of 604 nm emitting adenosine-capped QDs (at ˜100 nM) in DMEM cell culture medium to explore their suitability in intracellular imaging. To keep the reliability, we chose two cell lines, human ovarian cancer cell (HO-8910) and human liver cancer cell (SMMC-7721).

HO-8910 and SMMC-7721 cell lines were cultured overnight (37° C., 5% CO2) on glass chamber slides in Dulbecco's Modified Eaglets medium (DMEM) supplemented with 4 mM L-glutamine, 1 mM sodium pyruvate, 1% (v/v) penicillin/streptomycin/actinomycin D-antibiotic/antimyeotic, and 10% (v/v) heat- inactivated fetal bovine serum (FBS). QDs were adding into the culture medium (final concentration was 100 nM) and incubated for a certain time. Then removing from culture medium, cells were fixed with 2% paraformaldehyde, washed with PBS, followed by 4′,6-diamidino-2-phenylindole dihydrochloride (DAN) staining (18.7 uM) of the nuclear for 5 min, washed with PBS for three times, and finally immersed in 30% (v/v) glycerol/PBS. Fluorescence images were recorded with 450-550/405 nm and 550-650/488 nm emission/excitation for the visualization of DAPI and QDs respectively.

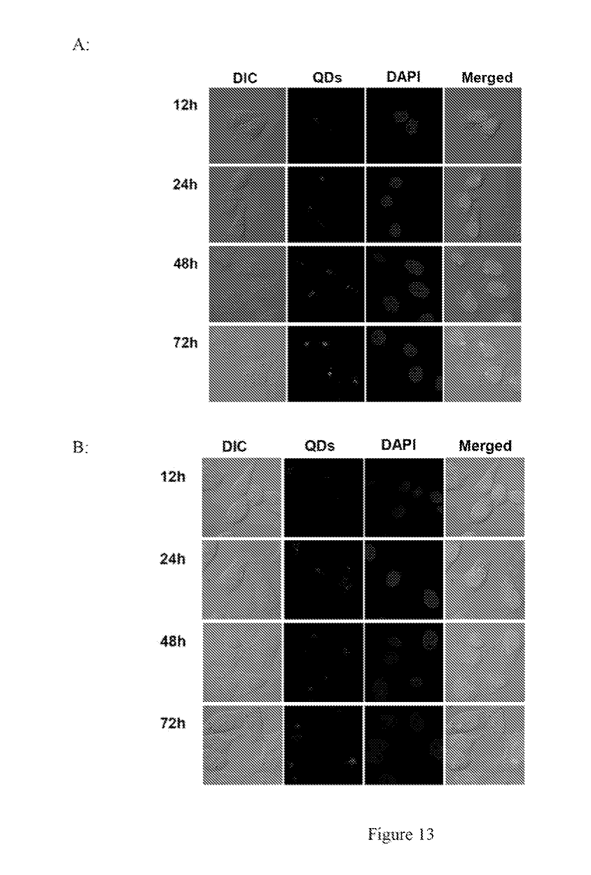

FIG. 13 shows the confocal microscopy of QDs endocytosed by HO-8910 (panel A) at different incubation times and the corresponding results for SNOW-7721 cells (panel B). Each row of image panels in FIG. 13 shows representative DIC, 604-nm emitting QDs (red), DAPI (blue), and the merged fluorescent composite images (right). For incubation of 12 hours, a substantial intracellular uptake of QDs took place as indicated by the pronounced fluorescence intensity measured for the cells. With prolonging incubation time, QDs maintained inner cells and stayed adjacent to the cell nucleoli.

It is notable that the adenosine-capped QDs were highly concentrated in one pole outside the nucleoli of the two kinds of cancer cells. This demonstrates the specific binding of QDs to the nuclear membrane, which is different from the commonly observed non-specific absorption feature of QDs in previous literature reports. No morphological and QDs PL brightness changes were observed even under incubation time up to 72 hours. This demonstrates that the adenosine-capped QDs were stable in cells and suitable for in vitro cell labeling, cell tracking, and other bioimaging applications. These promising features will enhance the performance of QDs as probes in many biological imaging applications, such as long-term single-molecule tracking or simultaneous multicolor imaging. It may also be used in other types of semiconducting or metal nanoparticles and may benefit in vivo applications, where small size and stability are crucial parameters for targeting and renal elimination.

1. A reversibly water-soluble nanocrystal comprising a core nanocrystal capped by a plurality of nucleotides, a plurality of nucleosides or a combination thereof.

2. The reversibly water-soluble nanocrystal of claim 1, wherein the core nanocrystal is a quantum dot, a nanorod, a metal oxide nanocube, or a noble metal nanodot.

3. The reversibly water-soluble nanocrystal of claim 1, wherein the core nanocrystal comprises a quantum dot having a core/shell/shell structure.

4.-6. (canceled)

7. The reversibly water-soluble nanocrystal of claim 1, wherein the plurality of nucleotides comprises adenosine 5′-monophosphate, guanosine 5′-monophosphate, cytidine 5′-monophosphate, uridine 5′-monophosphate, thymidine 5′-monophosphate, or combinations thereof, and wherein the plurality of nucleosides comprises adenosine, guanosine, cytidine, uridine, thymidine, inosine, or combinations thereof.

8. The reversibly water-soluble nanocrystal of claim 1, wherein the plurality of nucleotides comprises adenosine 5′-diphosphate, guanosine 5′-diphosphate, cytidine 5′-diphosphate, uridine 5′ -diphosphate, thymidine 5′ -diphosphate, adenosine 5′ -triphosphate, guanosine 5′-triphosphate, cytidine 5′-triphosphate, uridine 5′-triphosphate, thymidine 5′-triphosphate, or combinations thereof.

9. The reversibly water-soluble nanocrystal of claim 1, wherein the plurality of nucleotides comprises deoxynucelotides and wherein the plurality of nucleosides comprises deoxynucleosides.

10. The reversibly water-soluble nanocrystal of claim 1, further characterized by being soluble and stable in water for at least about sixty days without substantial aggregation or precipitation.

11. The reversibly water-soluble nanocrystal of claim 1, further characterized by retaining substantially the same size, morphology, and size distribution when dispersed in water as compared to the core nanocrystal when capped by hydrophobic ligands and dispersed in hydrophobic media.

12. The reversibly water-soluble nanocrystal of claim 1, further characterized by retaining substantially the same luminescence brightness and colloidal stability when dispersed in water as when dispersed in a hydrophobic media.

13. The reversibly water-soluble nanocrystal of claim 1, wherein the plurality of nucleotides, the plurality of nucleosides or the combination thereof that have capped the core nanocrystal form a layer that is equal to or less than about five nanometers thick.

14.-16. (canceled)

17. The reversibly water-soluble nanocrystal of claim 1, further characterized in that the water-solubility may be reversed by displacing the plurality of nucleotides and/or nucleotides that have capped the core nanocrystal with a plurality of hydrophobic capping ligands.

18. (canceled)

19. The reversibly water-soluble nanocrystal of claim, further characterized in that the conversion between water-solubility and water-insolubility may be carried out for at least ten cycles without significantly altering the performance characteristics of the nanocrystal.

20.-25. (canceled)

26. The reversibly water-soluble nanocrystal of claim 1, further characterized by retaining substantially the same luminescence brightness and colloidal stability within a biological cell when compared to outside a cell in aqueous media.

27.-33. (canceled)

34. A method for producing reversibly water-soluble nanocrystals, the method comprising the steps of:
(i) providing water insoluble nanocrystals comprising core nanocrystals capped by a plurality of initial hydrophobic ligands; and
(ii) contacting the water insoluble nanocrystals of step (i) with a plurality of nucleotides, a plurality of nucleosides, or a combination thereof, thereby replacing the initial hydrophobic ligands that cap the core nanocrystal with a cap of nucleotides, nucleosides, or a combination thereof, thus rendering the core nanocrystal reversibly water-soluble.

35. The method of claim 34, wherein the core nanocrystal is selected from the group consisting of a quantum dot, a nanorod, a metal oxide nanocube, and a noble metal nanodot.

36. The method of claim 34, wherein the plurality of nucleotides comprises adenosine 5′ -monophosphate, guano sine 5′ -monophosphate, cytidine 5′ -monophosphate, uridine 5′-monophosphate, thymidine 5′-monophosphate, or combinations thereof, and wherein the plurality of nucleosides comprises adenosine, guanosine, cytidine, uridine, thymidine, inosine, or combinations thereof.

37. The method of claim 34, wherein the plurality of nucleotides comprises adenosine 5′-diphosphate, guanosine 5′-diphosphate, cytidine 5′-diphosphate, uridine 5′-diphosphate, thymidine 5′-diphosphate, adenosine 5′-triphosphate, guanosine 5′-triphosphate, cytidine 5′-triphosphate, uridine 5′-triphosphate, thymidine 5′-triphosphate, or combinations thereof.

38. A method for reversibly transferring nanocrystals between a hydrophobic solution and a hydrophilic solution, the method comprising:
(i) providing a hydrophobic solution comprising core nanocrystals capped by a plurality of hydrophobic ligands;
(ii) mixing the hydrophobic solution with a solution comprising a plurality of nucleotides and/or nucleosides in order to replace the hydrophobic ligands capping the core nanocrystal with the nucleotides and/or nucleosides, thereby producing a ligand-exchanged solution comprising nucleotide- and/or nucleoside-capped nanocrystals; and
(iii) mixing the ligand-exchanged solution with a hydrophilic solution, whereby the nucleotide- and/or nucleoside-capped nanocrystals transfer from the hydrophobic, ligand-exchanged solution to the hydrophilic solution.

39.-40. (canceled)

41. The method of claim 38, wherein the core nanocrystal comprises a quantum dot, a nanorod, a metal oxide nanocube, a noble metal nanodot, or combinations thereof.

42. The method of claim 38, wherein the plurality of nucleotides comprises adenosine 5′ -monophosphate, guano sine 5′ -monophosphate, cytidine 5′ -monophosphate, uridine 5′-monophosphate, thymidine 5′-monophosphate, or combinations thereof, and wherein the plurality of nucleosides comprises adenosine, guanosine, cytidine, uridine, thymidine, inosine, or combinations thereof.

43. The method of claim 38, wherein the plurality of nucleotides comprises adenosine 5′-diphosphate, guanosine 5′-diphosphate, cytidine 5′-diphosphate, uridine 5′ -diphosphate, thymidine 5′-diphosphate, adenosine 5′ -triphosphate, guanosine 5′-triphosphate, cytidine 5′-triphosphate, uridine 5′-triphosphate, thymidine 5′-triphosphate, or combinations thereof.

44. The method of claim 38, further comprising:
(iv) mixing the hydrophilic solution with (a) a solution comprising a plurality of hydrophobic ligands and (b) a secondary hydrophobic solution in order to replace the nucleotides and/or nucleosides capping the core nanocrystal with the hydrophobic ligands, thereby producing a secondary ligand-exchanged solution comprising hydrophobic ligand-capped nanocrystals, whereby the hydrophobic ligand-capped nanocrystals transfer from the hydrophilic solution to the secondary hydrophobic solution.

45. The method of claim 44, wherein the hydrophobic ligands comprise organic amine ligands.

46.-47. (canceled)

48. A composition comprising a biological reagent coupled to a reversibly water-soluble nanocrystal comprising a core nanocrystal capped by a plurality of nucleotides, a plurality of nucleosides, or a combination thereof.

49. The composition of claim 48, wherein the biological reagent comprises an antibody, a nucleic acid probe, an enzyme substrate, a binding protein, or combinations thereof.

50. (canceled)

51. A method for labeling a biological cell or molecular component of such cell, the method comprising the step of:
contacting the cell or molecular component with a composition comprising a biological reagent coupled to a reversibly water-soluble nanocrystal comprising a core nanocrystal capped by a plurality of nucleotides, a plurality of nucleosides, or a combination thereof.

52.-54. (canceled)

55. A biological cell or molecular component of such cell labeled with a composition comprising a biological reagent coupled to a reversibly water-soluble nanocrystal comprising a core nanocrystal capped by a plurality of nucleotides, a plurality of nucleosides, or a combination thereof.

56. The biological cell or molecular component of such cell of claim 55, wherein the biological cell is a cancer cell.

57. The biological cell or molecular component of such cell of claim 55, wherein the molecular component of such cell is a nuclear membrane.
